(19) United States
(12) Patent Application Publication
(10) Pub. No.: US 2008/0044350 A1
(43) Pub. Date: Feb. 21, 2008

(54) Optical Imaging Contrast Agents for Imaging Lung Cancer

(76) Inventors: Jo Klaveness, Oslo (NO); Edvin Johannesen, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/582,842

(22) PCT Filed: Dec. 17, 2004
(86) PCT No.: PCT/NO04/00394

§ 371 Date: May 2, 2007

(30) Foreign Application Priority Data
Dec. 18, 2003 (NO) 20035683

(51) Int. Cl. A61K 49/00; A61P 35/00

(52) U.S. Cl. 424/9.1

(57) ABSTRACT

The invention provides contrast agents for optical imaging of prostate cancer in patients. The contrast agents may be used in diagnosis of prostate cancer, for follow up of progress in disease development, for follow up of treatment of prostate cancer and for surgical guidance. Further, the invention provides methods for optical imaging of prostate cancer in patients.

### FIELD OF THE INVENTION

The present invention provides contrast agents for optical imaging of prostate cancer in patients. The contrast agents may be used in diagnosis of prostate cancer, for follow up of progress in disease development, and for follow up of treatment of prostate cancer.

The present invention also provides new methods of optical imaging of prostate cancer in patients, for diagnosis and for follow up of disease development and treatment of prostate cancer.

### DESCRIPTION OF RELATED ART

Prostate cancer is the most common cancer type for men in the western world. More than 25% of all men with a cancer diagnosis have prostate cancer.

The prostate is a gland with the size of a walnut located between the rectum and the bladder. The gland stores a fluid that is released to form semen. The function of the prostate gland is dependent on testosterone.

Tumours in the prostate gland can be benign or malignant. Benign prostatic hyperplasia (BPH) is not a cancer but is an abnormal growth of prostate cells. The result is that the prostate gland increases in size and presses against the urinary system resulting in problems with urination. BPH is very common in elderly men. BPH is a disease that normally does not require treatment and the disease is not fatal. Prostate cancer, on the other hand is as most other forms of cancer fatal if not treated.

Prostate cancer is usually diagnosed in elderly men at the age of about 70 years. Risk factors for prostate cancer include race, the disease being more common for African men than for white men, diet factors and family history.

In its early state, prostate cancer does not cause symptoms. The main symptoms of prostate cancer, however, can include: problems related to urination, such as pain, weak flow of urine and frequent urination. Other symptoms include difficulty in obtaining erection, blood in urine or semen, painful ejaculation and lower back pain.

The simplest test to perform to indicate prostate cancer is a blood test for prostate-specific antigen (PSA). The PSA level in blood may increase as a result of prostate cancer; however PSA levels may also increase in patients with other diseases of the prostate. Rectal examination of the prostate to check for size and hardness of the prostate is also a simple test to diagnose prostate disease. Other tests for prostate cancer include blood level measurements of prostatic acid phosphatase (PAP), biopsy and imaging techniques like cystoscopy, X-ray and transrectal ultrasonography.

Staging of prostate cancer can be a difficult task. Normally prostate cancer can be separated into four different stages (I-IV). At stage I the cancer cannot be detected by rectal examination, at stage II the cancer does not include other tissue than prostate tissue, at stage III the disease has spread to nearby tissue and at stage IV the disease has spread to the lymph nodes.

As for all cancer forms, early diagnosis is essential in obtaining a good result in treatment of prostate cancer. The main treatments of prostate cancer include surgery, radiation therapy and drugs, mainly hormonal therapy.

Drug therapy of prostate cancer includes antiandrogens like flutamide and bicalutamide, luteinizing hormone releasing agents like bruserelin and leuprolide and drugs like ketoconazole and aminoglutethimide. The drug called finasteride might be useful to prevent prostate cancer. Development of improved methods for early diagnosis of prostate cancer will improve the therapeutic outcome in treatment of the disease. Several groups have therefore suggested various methods for diagnosis of prostate cancer:

Blood tests exist wherein concentrations of e.g. prostate-specific antigen (PSA) or other biomarkers are measured. See e.g. U.S. Pat. No. 6,309,816 (Horus Therapeutics) which is directed to a method of screening for prostate cancer by measuring the concentration of PSA and creatine kinase enzyme activity.

Some in vitro methods have been described identifying or measuring concentrations of specific biomarkers in prostate tissue. See e.g. U.S. Pat. No. 5,824,490 (John Hopkins University) which relates to an in vitro method for detecting prostate cancer based on prostate cancer-1 protein. U.S. Pat. No. 6,479,263 (Baylor College of Medicine) relates to detection of prostate cancer by determining the presence of hK2 RNA in a physiological sample.

Other methods that have been described are directed to gene tests. See e.g. U.S. Pat. No. 5,506,106 (Thomas Jefferson) which relates to methods of detecting micrometastasis of prostate cancer based on RNA. U.S. Pat. No. 6,569,684 (University of Central Florida) describes a method of determining human prostate cancer in a non-invasive way using prostasin gene promoter DNA methylation level, sampling a human prostate carcinoma tissue.

Some in vivo diagnostic methods using contrast agents have been described. See e.g. U.S. Pat. No. 6,428,479 (Amersham Health AS) which describes a method for diagnosis of prostate cancer abnormalities including prostate cancer using ultrasound contrast agents. U.S. Pat. No. 6,517,811 and U.S. Pat. No. 5,911,970 (Research Corporation Technologies) relate to radionuclide based contrast agents for imaging and therapy of cancer, including prostate cancer.

There is today no reliable method for early diagnosis of prostate cancer. PSA and other biomarkers are not selective for cancer, and existing in vivo imaging methods are not sensitive enough to diagnose prostate cancer at an early stage.

As pointed out prostate cancer is still a challenge to diagnose and treat. There is a need for improved diagnostic methods, especially for diagnosis of prostate cancer in an early stage with good reliability. Surprisingly we have discovered that the use of optical imaging methods with new optical imaging contrast agents fulfil these requirements.

### SUMMARY OF THE INVENTION

The present invention provides an optical imaging contrast agent with affinity for an abnormally expressed biological target associated with prostate cancer.

The invention is also described in the claims.

The following definitions will be used throughout the document:

Prostate cancer tissue: Any tissue in the prostate that shows changes associated with neoplasia or pre-neoplasia, and including metastases from prostate cancer at other sites in the body.

Abnormally expressed target: A target that is either overexpressed or downregulated in prostate cancer tissue.

Overexpressed target: A receptor, an enzyme or another molecule or chemical entity that is present in a higher amount in prostate cancer tissue than in normal tissue.

Downregulated target: A receptor, an enzyme or another molecule or chemical entity that is present in a lower amount in prostate cancer tissue than in normal tissue.

### DETAILED DESCRIPTION OF THE INVENTION

A first aspect of the present invention is an optical imaging contrast for imaging of prostate cancer. By the term optical imaging contrast agent, or just contrast agent, we mean a molecular moiety used for enhancement of image contrast in vivo comprising at least one moiety that interacts with light in the ultraviolet, visible or near-infrared part of the electromagnetic spectrum.

The contrast agent has affinity for an abnormally expressed target associated with prostate cancer.

Prostate cancer tissue containing a downregulated target is identified by a low amount of bound imaging agent compared to normal tissue. In this situation, the amount of imaging agent should be less than 50% of that in normal tissue, preferably less than 10%.

Preferred contrast agents according to the invention, have affinity for an overexpressed target associated with prostate cancer. Preferred targets are those targets that are more than 50% more abundant in prostate cancer tissue than in surrounding tissue. More preferred targets are those targets that are more than two times more abundant in prostate cancer tissue than in surrounding tissue. The most preferred targets are those targets that are more than 5 times more abundant in prostate cancer tissue than in surrounding tissue.

In a further aspect of the invention, targets that are mutated in prostate cancer tissue are identified by lack of binding of an imaging agent that does bind to normal tissue; alternatively, the imaging agent might be directed specifically towards the mutated target, and binding to normal tissue would be minimal. The mutated target can be a protein in prostate cancer tissue that is altered as a result of a germline or somatic mutation, and including alterations resulting from differential splicing of RNA and changes in post-translational modifications, particularly glycosylation patterns, but not limited to these types of alterations.

Relevant groups of targets are receptors, enzymes, nucleic acids, proteins, lipids and other macromolecules as, for example, lipoproteins and glycoproteins. The targets may be located in the vascular system, in the extracellular space, associated with cell membranes or located intracellularly.

Preferred groups of targets are adhesion molecules, extracellular matrix and related proteins, antigens, cell cycle control proteins, enzymes and inhibitors, hormones and cytokines, hormone receptors and related proteins, signal transducing proteins and transcription factors and related proteins associated with prostate cancer.

The following biological targets are overexpressed in prostate cancer and are preferred targets for optical imaging contrast agent of the invention:

Adhesion Molecules, Extracellular Matrix and Related Proteins

Beta-catenin, delta-catenin, CD44, hyaluronic acid, hyaluronidase, E-cadherin (ECAD; cell adhesion molecule), alpha-catenin, beta-catenin, delta-catenin, gamma-catenin, p120, syndecan-1, galectin 1 and integrins such as αᵥβ₃ and αᵥβ₅.

Antigens

Alpha-fetoprotein, immunoglobulin G, immunoglobulin M, 65 kDa oncofetal protein, p65, prostate stem cell antigen (PSCA), Ki-67, CD 11 b, CD 14, PSA oligosaccharides, prostate secretory protein PSP-94, Ca 15-3, prostate specific membrane associated antigen (PSMA) and prostate cancer specific antigen.

Cell Cycle Control Proteins

Bax, Bax inhibitor-1, Cdc42, CHEK2, cyclin A, Cyclin B1, HMGI(Y), p53, survivin, truncated form of Bcl-2 (Bcl-2psi), Bcl-2.

Enzymes and Inhibitors

25-hydroxyvitamin D-1alpha-hydroxylase, alpha-1-antichymotrypsin, 2′-5′-oligoadenylate-dependent ribonuclease L (RNASEL), testosterone 5alpha-reductase, acetyl-CoA-carboxylase, Akt kinase, alpha-methylacyl-CoA racemase (AMACR), bone alkaline phosphatase, cathepsin D, CD-10 (neutral endopeptidase), cyclooxygenase 2, cystatin A, endometase/matrilysin-2/matrix metalloproteinase-26 (MMP-26), enzymes of ceramide metabolism, e.g. acid ceramidase, Enzymes in polyamine metabolism, farnesyl diphosphate synthase, fatty acid synthase (FASE), furin, glandular kallikrein 2 (hK2), glutathione S-transferase, gp91 phox, gp91 phox homolog NOX5, GSK-3 beta, hepsin, HMG-CoA-reductase, farnesyl diphosphate synthase, Human kallikrein 11 (hK11/trypsin-like serine protease/TLSP, human kallikrein 2, Integrin-linked kinase (ILK), Kallikrein 10 (KLK10), kallikrein 13 (hK13), Kinase-associated phosphatase (KAP), LIM kinase 1, Lipoxygenase -5, manganese superoxide dismutase (SOD-2), matrix metalloproteinases, particularly MMP-2, MMP-9, and MT1-MMP (MMP-14), N-acetylated alpha-linked acidic dipeptidase (NAALADase), N-acetyltransferase-2 (NAT2), neuron-specific enolase (NSE), p22phox, phosphatidylinositol-4-phosphate 5-kinase type I alpha (PIP5KIA), pim-1 kinase, prostate-specific antigen (PSA), prothrombin activation peptide F(1+2) (IF2), PTEN, SCFSkp2 ubiquitin ligase, steroid 5 alpha-reductase type II (SRD5A2), human sterol isomerase (HSI), telomerase-associated protein, testosterone 5alpha-reductase, Thymidine phosphorylase (TP), urokinase plasminogen activator (uPA), urokinase receptor (uPAR), COX -2.

Hormones and Cytokines

Fibroblast growth factor 5, inhibitory insulin-like growth factor binding protein (IGFBP), thymosine beta 15, Transforming growth factor-beta (TGF-beta), sigma 1 receptor, insulin-like growth factor binding protein 2 (IGFBP2), fibroblast growth factor 8 (FGF8), netrin, Insulin-like growth factor-I (IGF-I), IGF binding protein-3, growth hormone-releasing hormone (GHRH), chorionic gonadotropin (CG), bombesin (BBS)-like peptide, gastrin-releasing peptide (GRP), vascular endothelial growth factor (VEGF), interleukin-6 (IL-6), LHRH, macrophage inhibitory cytokine-1 (MIC-1), parathyroid hormone-related peptide, Mac25/insulin-like growth factor binding-protein-related protein-1, leptin, stromal-derived factor-1 (SDF-1 or CXCL12), transforming growth factor beta1, macrophage inhibitory cytokine 1, insulin-like growth factor (IGF), insulin-like growth factor I (IGF-I), Insulin-like growth factor binding protein 2 (IGFBP2), insulin-like growth factor binding protein 3 (IGFBP-3), progastrin-releasing peptide.

Hormone Receptors and Related Proteins

Osteoprotegerin (OPG), endothelin B receptor, androgen receptor (AR), DCC (Deleted in Colorectal Cancer) protein, gastrin-releasing peptide receptor GRP-R, LHRH receptor, interleukin (IL) 10/IL-10 receptor, receptor CXCR4, bradeion, androgen receptor, epidermal growth factor receptor (EGFR, her-2/neu), Cdc 25B (an androgen receptor coactivator), 67-kD laminin receptor, vascular endothelial growth factor receptor (VEGFR) and c-met.

Signal Transducing Proteins

EphA2 tyrosine kinase, HER2, extracellular signal-regulated kinase (ERK)(MAPK)/p38(SAPK), transient receptor potential p8 (trp-p8), voltage-gated Na(+) channels (VGSCs), purinergic receptor, N-my, Alpha IH (Cav 3.2) T-type calcium channel, PIM-1.

Transcription Factors and Related Proteins

Tat interactive protein, 60 kDa (Tip60), nuclear factor (NF) kappaB, NFATc1, GAGATA-binding Protein, EZH2 (enhancer of zeste homolog 2).

Others

34 beta E12 cytokeratin, androgen-regulated prostate specific gene (NKX3.1) product, apolipoprotein D (apoD), apolipoprotein E, calcium binding protein S100A4, caveolin-1, chromogranin A (CgA), CK 14, CK 5/6, cytokeratins (particularly cytokeratins 8, 14, 15, 18, 19 and 20), elongin C, Fas (Apo-1/CD95), Fas ligand (FasL), FK506-binding protein 51 (FKBP51), GRP78 (glucose-regulated protein 78 kDa), HGF/SF, hK2, HPV-16 E, HSP10, HSP60, huntingtin-interacting protein 1, IMP1 , Koc, Krüppel-like factor 6, Macrophage Scavenger Receptor 1 (MSR1), Osteocalcin, p16, p63, p70S6K, Part-1 protein, PCNA, platelet factor 4 protein (PF4), Prostate derived factor (PDF), PSP94 (prostate secretory protein 94), PSP61 (prostate secretory protein 61), PTOV1 protein, villin, Von Willebrand factor, prostate cancer specific gene PCGEM1 protein.

More preferred markers that are overexpressed in prostate cancer are: Steroid 5 alpha-reductase (5AR), fatty acid synthase, COX-2, prostate specific antigen, androgen receptor, Lipoxygenase-5, vascular endothelial growth factor receptor (VEGFR), Cyclin D1, CD44, Syndecan-1, prostate stem cell antigen (PSCA), Ki-67, alpha-methylacyl-CoA racemase (AMACR), cathepsin D, hepsin, MT1-MMP, urokinase receptor (uPAR), androgen receptor (AR), epidermal growth factor receptor (EGFR/her-2/neu), Fas (Apo-1/CD95), Fas ligand (FasL) and Macrophage Scavenger Receptor 1 (MSR1).

The most preferred targets that are overexpressed in prostate cancer are COX-2, prostate-specific antigen, MT1-MMP, urokinase receptor, epidermal growth factor receptor and vascular endothelial growth factor receptor (VEGFR).

There are also targets that are downregulated in prostate cancer. These are beta-catenin, tenascin, Stat5a, cyclin D1, p27, CDKN1B protein, cell cycle inhibitor p27, effector PKAc, Raf kinase inhibitor protein (RKIP), CD10, cystatin B, cyclin-dependent kinase inhibitor, 27(Kip1), TGF-beta type II receptor (T beta R II), NKCC1 protein, calcium binding protein S100A2, prostasin, annexins 1, 2, 4, 7, and 11, A3 adenosine receptor (A3AR), c-myc, NF-kappa B/p65, Creatine kinase, Steroid 5 alpha-reductase, hevin, gelsolin and galactin-3.

Preferred targets that are downregulated in prostate cancer are:

Tenascin, TGF-beta type II receptor (T beta R II), A3 adenosine receptor (A3AR), steroid 5 alpha-reductase.

Generally, any targets that have been identified as possible targets for agents for treatment of prostate cancer are potential targets also in optical imaging.

The preferred contrast agents of the present invention are molecules with relatively low molecular weights. The molecular weight of preferred contrast agents is below 14 000 Daltons, preferably below 10000 Daltons and more preferably below 7000 Daltons.

The contrast agents are preferably comprised of a vector that has affinity for an abnormally expressed target in prostate cancer tissue, and an optical reporter.

Thus viewed from one aspect the present invention provides a contrast agent of formula I:

V-L-R (I)

wherein V is one or more vector moieties having affinity for one or more abnormally expressed target in prostate cancer tissue, L is a linker moiety or a chemical bond and R is one or more reporter moieties detectable in optical imaging.

The vector has the ability to direct the contrast agent to a region of prostate cancer. The vector has affinity for the abnormally expressed target and preferably binds to the target. The reporter must be detectable in an optical imaging procedure and the linker must couple vector to reporter, at least until the reporter has been delivered to the region of prostate cancer and preferably until the imaging procedure has been completed.

The vector can generally be any type of molecules that have affinity for the abnormally expressed target. The molecules should be physiologically acceptable and should preferably have an acceptable degree of stability. The vector is preferably selected from the following group of compounds: peptides, peptoids/peptidomimetics, oligonucleotides, oligosaccharides, lipid-related compounds like fatty acids, traditional organic drug-like small molecules, synthetic or semi-synthetic, and derivatives and mimetics thereof. When the target is an enzyme the vector may comprise an inhibitor of the enzyme or an enzyme substrate. The vector of the contrast agent preferably has a molecular weight of less than 10 000 Daltons, more preferably less than 4500 Daltons and most preferably less than 2500 Daltons, and hence does not include antibodies or internal image antibodies. In addition to problems with immune reactions, long circulation time and limited distribution volume, many antibodies have an affinity for the receptor that is too low for use in imaging.

An optical imaging contrast agent comprising a vector having affinity for any of the preferred targets is a preferred embodiment of the invention.

Contrast agents having affinity for more than one abnormally expressed target related to the disease is an aspect of the invention. Such contrast agents can comprise two or more different vectors or molecular subunits that target two or more different abnormally expressed targets.

Another possibility according to the present invention is that the contrast agent comprises one vector that is able to bind to more than one abnormally expressed target in prostate cancer.

A contrast agent according to the present invention can also comprise more than one vector of same chemical composition that bind to the abnormally expressed biological target.

Some receptors are unique to endothelial cells and surrounding tissues. Examples of such receptors include growth factor receptors such as VEGF and adhesion receptors such as the integrin family of receptors. Peptides comprising the sequence arginine-glycine-aspartic acid (RGD) are known to bind to a range of integrin receptors. Such RGD-type peptides constitute one group of vectors for targets associated with prostate cancer.

Below are some examples of vectors having affinity for prostate cancer-related abnormally expressed targets:

Vectors for cyclo-oxygenase-2 (COX-2):

Arachidonic acid [506-32-1], (Sigma A9673, A8798):

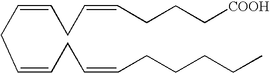

Arachidonic acid is the endogenous substrate for COX-2, and is an essential fatty acid and a precursor in the biosynthesis of prostaglandins.

Other vectors for COX-2 are exogenous compounds that bind to COX-2, for example so-called COX-2 inhibitors. The chemical classes of the main COX-2 inhibitors are shown in WO 02/07721.

Such vectors include:

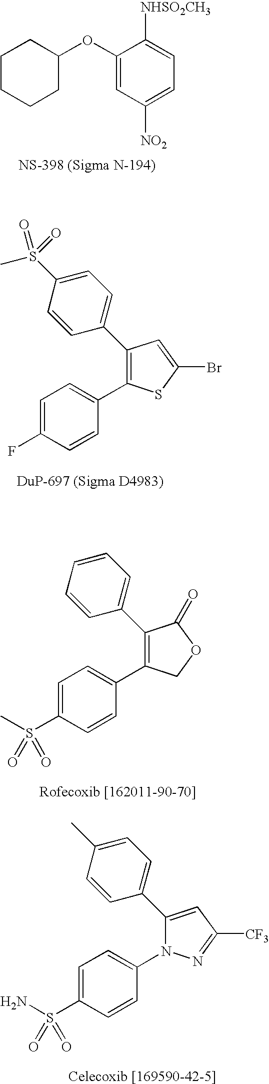

Vectors for Steroide 5-α Reductase (5AR):

The 5AR enzyme reduces testosterone to dihydrotestosterone (DHT). DHT is a 10 times more potent androgen than testosterone.

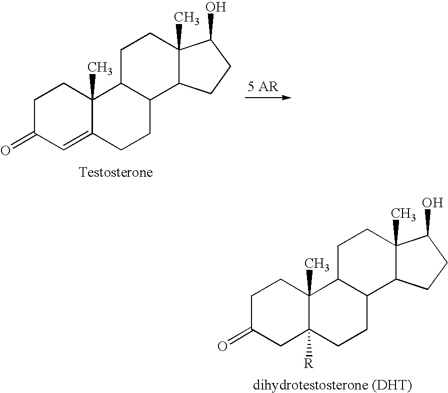

The following potential vectors are 5-α reductase inhibitors:

Finasterid [98319-26-7]:

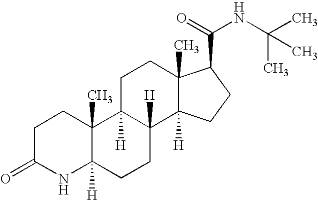

Benzoquinolinones:

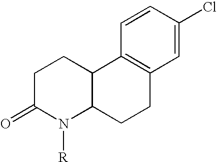

Indole acid derivatives:

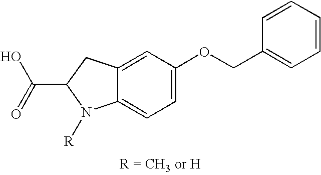

Polyphenols:

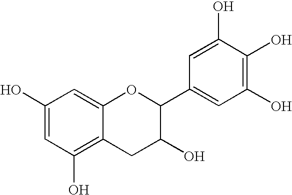

Vectors for Androgen Receptors:

Testosterone (agonist):

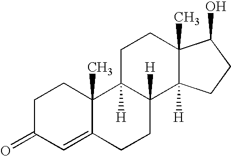

5-α-dihydrotestosterone (agonist):

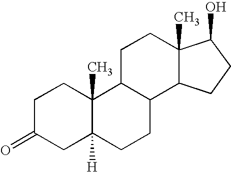

Cyproteron (antagonist) [2098-66-01]:

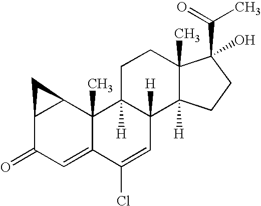

Mibolerone [3704-09-04]:

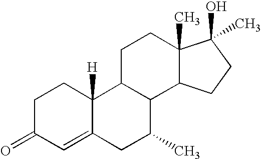

Methyltrienolone [965-93-5]:

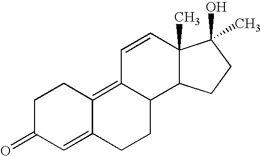

A wide variety of linkers can be used. The linker component of the contrast agent is at its simplest a bond between the vector and the reporter moieties. In this aspect the reporter part of the molecule is directly bound to the vector that binds to the abnormally expressed target. More generally, however, the linker will provide a mono- or multi-molecular skeleton covalently or non-covalently linking one or more vectors to one or more reporters, e.g. a linear, cyclic, branched or reticulate molecular skeleton, or a molecular aggregate, with in-built or pendant groups which bind covalently or non-covalently, e.g. coordinatively, with the vector and reporter moieties. The linker group can be relatively large in order to build into the contrast agent optimal size or optimal shape or simply to improve the binding characteristics for the contrast agent to the abnormally expressed target in prostate cancer tissue.

Thus linking of a reporter unit to a desired vector may be achieved by covalent or non-covalent means, usually involving interaction with one or more functional groups located on the reporter and/or vector. Examples of chemically reactive functional groups which may be employed for this purpose include amino, hydroxyl, sulfhydroxyl, carboxyl and carbonyl groups, as well as carbohydrate groups, vicinal diols, thioethers, 2-aminoalcohols, 2-aminothiols, guanidinyl, imidazolyl and phenolic groups.

The reporter is any moiety capable of detection either directly or indirectly in an optical imaging procedure. The reporter can be a light scatterer (e.g. a coloured or uncoloured particle), a light absorber or a light emitter. More preferably the reporter is a dye such as a chromophore or a fluorescent compound. The dye part of the contrast agent can be any dye that interacts with light in the electromagnetic spectrum with wavelengths from the ultraviolet light to the near-infrared. Preferably, the contrast agent of the invention has fluorescent properties.

Preferred organic dye reporters include groups having an extensive delocalized electron system, eg. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B, rhodamine 6G, rhodamine 19, indocyanine green, Cy2, Cy3, Cy3.5, Cy3.5B, Cy5, Cy5.5, Cy7, Cy7.5, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. The cyanine dyes are particularly preferred.

Particularly preferred are dyes which have absorption maxima in the visible or near-infrared region, between 400 nm and 3 μm, particularly between 600 and 1300 nm.

The contrast agents according to the invention can comprise more than one dye molecular sub-unit. These dye sub-units can be similar or different from a chemical point of view. Preferred contrast agents have less than 6 dye molecular sub-units.

Several relevant targets for prostate cancer are enzymes. A contrast agent for optical imaging of prostate cancer for targeting an enzyme can be an enzyme contrast agent substrate that can be transformed to a contrast agent product possessing different pharmacokinetic and/or pharmacodynamic properties from the contrast agent substrate. This embodiment of the invention provides contrast agent substrates having affinity for an abnormally expressed enzyme, wherein the contrast agent substrate changes pharmacodynamic and/or pharmacokinetic properties upon a chemical modification into a contrast agent product in a specific enzymatic transformation, and thereby enabling detection of areas of disease upon a deviation in the enzyme activity from the normal. Typical differences in pharmacodynamic and/or pharmacokinetic properties can be binding properties to specific tissue, membrane penetration properties, protein binding and solubility properties.

Alternatively, if the abnormally expressed target for diagnosis of prostate cancer is an enzyme, the contrast agent for optical imaging can be a dye molecule that directly binds to the enzyme. The contrast agent will have affinity for the abnormally expressed enzyme, and this may be used to identify tissue or cells with increased enzymatic activity.

In a further aspect of the invention, the contrast agent changes dye characteristics as a result of an enzymatic transformation. For example, a fluorescent dye reporter of the contrast agent is quenched (no fluorescence) by associated quencher groups, until an enzymatic cleavage takes place, separating the dye from the quencher groups and resulting in fluorescence at the site of the abnormally expressed enzyme.

Another aspect of this part of the invention is that the dye may change colour, as e.g. a change in absorption and/or emission spectrum, as a result of an enzymatic transformation.

If the abnormally expressed target for diagnosis of prostate cancer is a receptor or another non-catalytic target, the contrast agent for optical imaging can bind directly to the target and normally not change the dye characteristics.

The preferred contrast agents of the present invention are soluble in water. This means that the preferred contrast agents have a solubility in water at pH 7.4 of at least 1 mg/ml.

The contrast agents of the present invention can be identified by random screening, for example by testing of affinity for abnormally expressed targets of a library of dye labelled compounds either prepared and tested as single compounds or by preparation and testing of a mixture of compounds (a combinatorial approach). Alternatively, random screening may be used to identify suitable vectors, before labelling with a reporter.

The contrast agents of the present invention can also be identified by use of technology within the field of intelligent drug design. One way to perform this is to use computer-based techniques (molecular modelling or other forms of computer-aided drug design) or use of knowledge about natural and exogenous ligands (vectors) for the abnormally expressed targets. The sources for exogenous ligands can for example be the chemical structures of therapeutic molecules for targeting the same target. One typical approach here will be to bind the dye chemical sub-unit (reporter) to the targeting vector so that the binding properties of the vector are not reduced. This can be performed by linking the dye at the far end away from the pharmacophore centre (the active targeting part of the molecule).

The contrast agents of the invention are preferably not endogenous substances alone. Some endogenous substances, for instance estrogen, have certain fluorescent properties in themselves, but they are not likely to be sufficient for use in optical imaging. Endogenous substances combined with an optical reporter however, fall within the contrast agents of the invention.

The contrast agents of the invention are intended for use in optical imaging. Any method that forms an image for diagnosis of disease, follow up of disease development or for follow up of disease treatment based on interaction with light in the electromagnetic spectrum from ultraviolet to near-infrared radiation falls within the term optical imaging. Optical imaging further includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

Examples of contrast agents for optical imaging of prostate cancer according to the invention are shown below:

Contrast Agents with Affinity for COX-2:

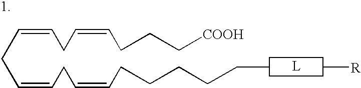

Wherein arachidonic acid, the endogenous substrate for COX-2, is coupled to a reporter (R) via a linker (L).

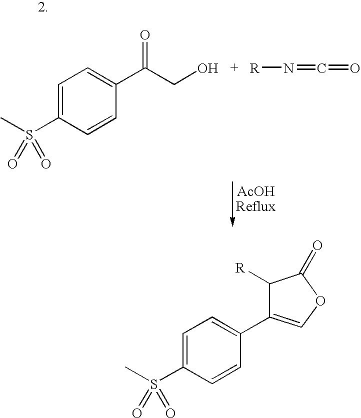

Wherein a COX-2-inhibitor derivative is linked to a reporter (R). R is any reporter according to the present invention; for example fluorescein, and L is a linker. For this example, giving a Rofecoxib-derivative, a possible synthesis is given.

Synthesis of Contrast Agents with Affinity for 5-α Reductase:

Contrast agent I: Linking the reporter Cy5.5 to an indole acid derivative.

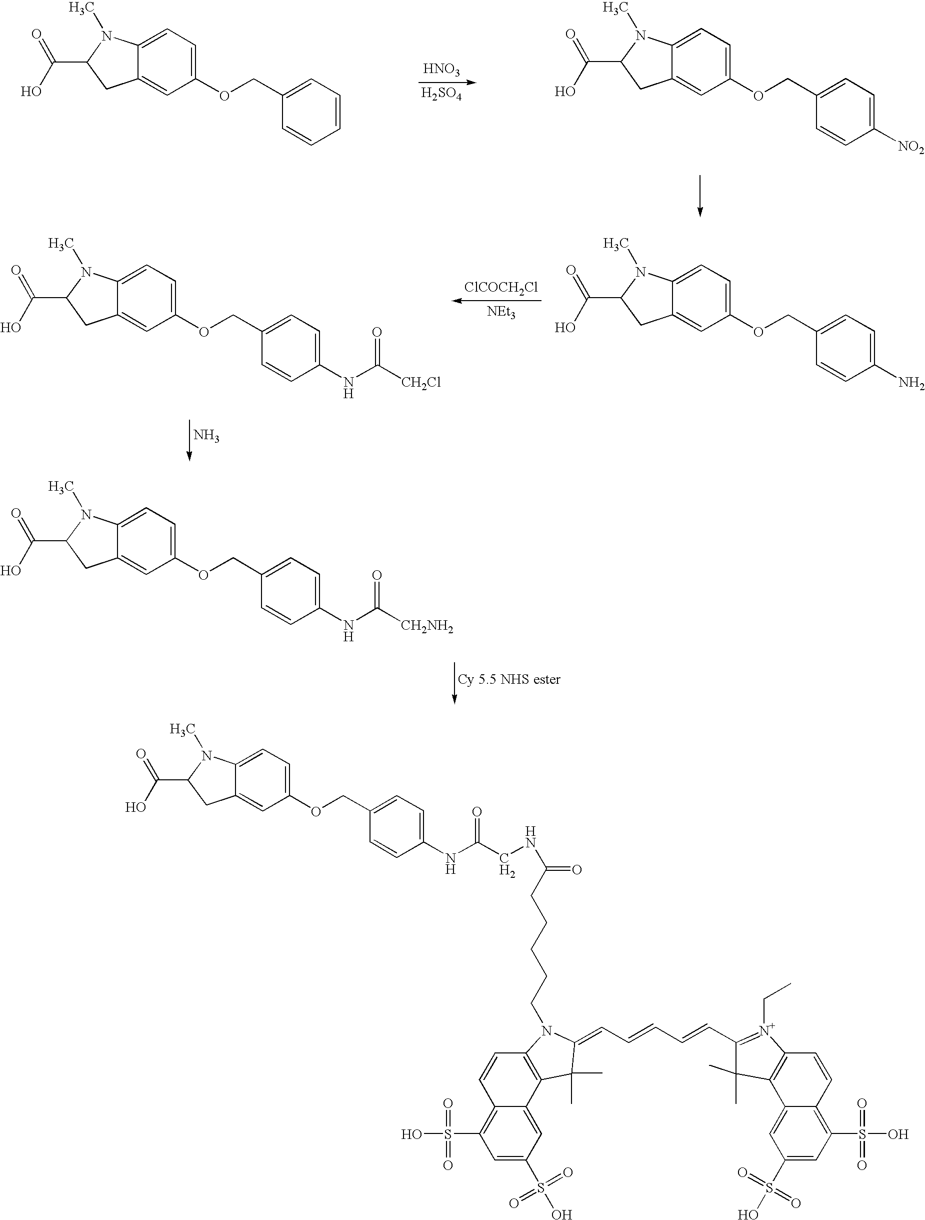

Contrast agent II:

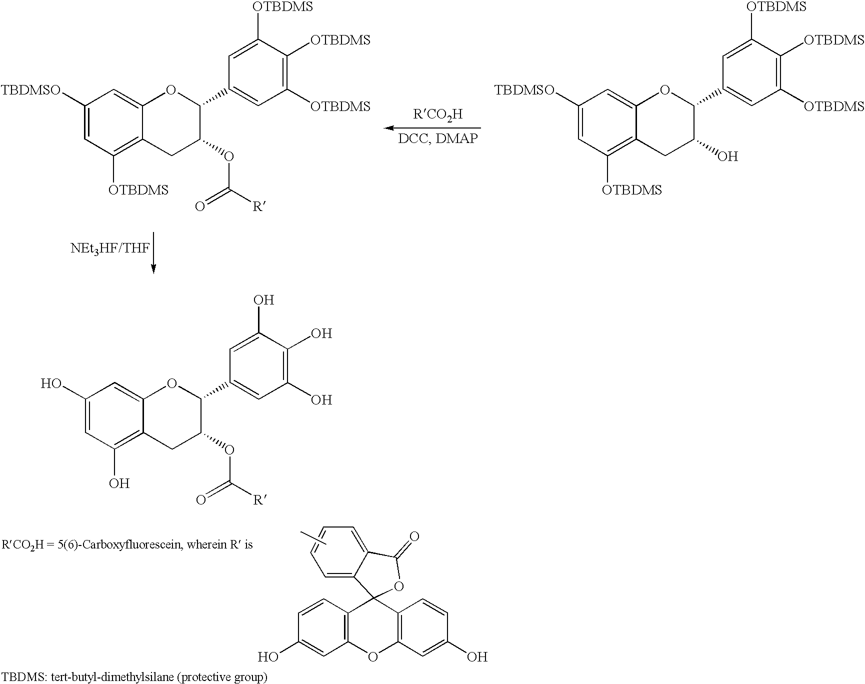

The starting material has been described by J. J. Plattner et al in JACS (19) 94 8613-5.

Contrast Agents with Binding Affinity to Androgen Receptors:

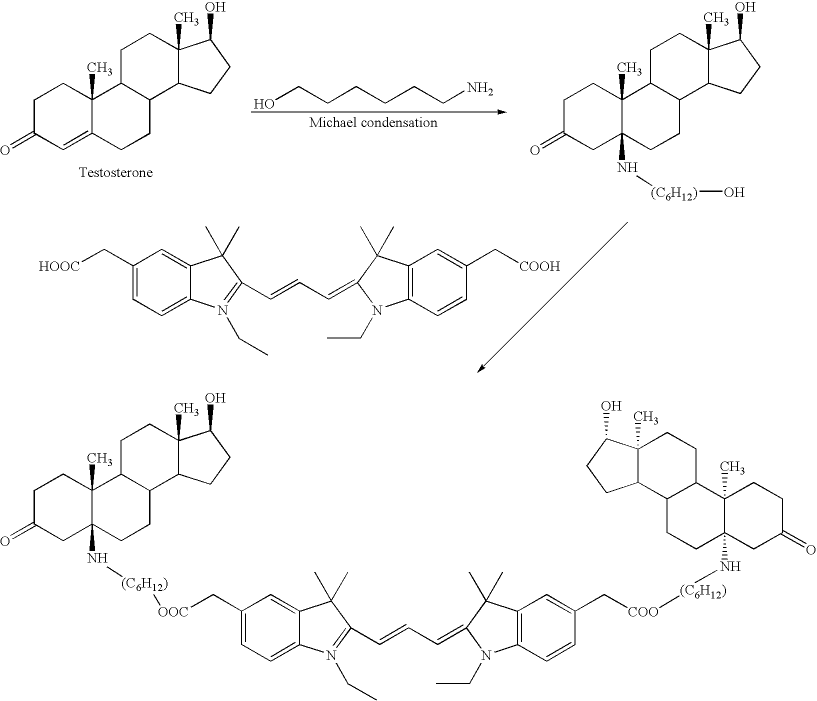

Wherein two groups of testosterone are linked to a NIR dye.

A further embodiment is the use of contrast agents of the invention for optical imaging of prostate cancer, that is for diagnosis of prostate cancer, for use in follow up the progress in prostate cancer development, for follow up the treatment of prostate cancer, or in surgical guidance.

In the context of this invention, diagnosis includes screening of selected populations, early detection, biopsy guidance, characterisation, staging and grading. Follow up of treatment includes therapy efficacy monitoring and long-term follow-up of relapse. Surgical guidance includes tumour margin identification during resection.

Still another embodiment of the invention is a method of optical imaging of prostate cancer using the contrast agents as described.

Still another embodiment of the invention is a method of optical imaging for diagnosis, to follow up the progress of prostate cancer development, to follow up the treatment of prostate cancer, or in surgical guidance, using the contrast agents as described.

One aspect of these methods is to administer the present contrast agents and follow the accumulation and elimination directly visually during surgery. Another aspect of these methods is to administer the present contrast agents and perform visual diagnosis through a colonoscope.

Still another aspect of the present invention is to administer the present contrast agents and perform the image diagnosis using computerized equipment as for example a tomograph.

Still another embodiment of the invention is use of a contrast agent as described for the manufacture of a diagnostic agent for use in a method of optical imaging of prostate cancer involving administration of said diagnostic agent to an animate subject and generation of an image of at least part of said body, preferably the prostate gland or parts of the prostate gland.

Still another embodiment of the invention is pharmaceutical compositions comprising one or more contrast agents as described or pharmaceutically acceptable salts thereof for optical imaging for diagnosis of prostate cancer, for follow up progress of prostate cancer development or for follow up the treatment of prostate cancer. The contrast agents of the present invention may be formulated in conventional pharmaceutical or veterinary parenteral administration forms, e.g. suspensions, dispersions, etc., for example in an aqueous vehicle such as water for injections. Such compositions may further contain pharmaceutically acceptable diluents and excipients and formulation aids, for example stabilizers, antioxidants, osmolality adjusting agents, buffers, pH adjusting agents, etc. The preferred formulation is a sterile solution for intravascular administration or for direct injection into area of interest. Where the agent is formulated in a ready-to-use form for parenteral administration, the carrier medium is preferably isotonic or somewhat hypertonic.

The dosage of the optical contrast agent diagnostic agents of the invention will depend upon the clinical indication, choice of contrast agent and method of administration. In general, however dosages will be between 1 micro gram and 70 grams and more preferably between 10 micro grams and 5 grams for an adult human.

While the present invention is particularly suitable for methods involving parenteral administration of the contrast agent, e.g. into the vasculature or directly into an organ or muscle tissue, intravenous administration being especially preferred, it is also applicable where administration is not via a parenteral route, e.g. where administration is transdermal, nasal, sub-lingual or is into an externally voiding body cavity, e.g. the colon, rectum or bladder. The present invention is deemed to extend to cover such administration.

The following examples are illustrative only and not intended to be limiting. Other features and advantages of the invention will be apparent from the detailed description and from the claims.

### EXAMPLES

### Example 1

### Contrast Agent for Mapping of COX-2 Activity. Synthesis of COX-2 Ligand Coupled to Fluorescein

Step 1

2-Hydroxy-1-(4-methanesulfonylphenyl)ethanone is prepared from 2-bromo-1-(4-methanosulfonylphenyl)ethanone according to C. Puig et al in J. Med. Chem 2000,43, 214-223.

Step 2

A solution of 2-hydroxy-1-(4-methanosulfonylphenyl) ethanone (1.50 g, 7 mmol) and fluorescein isocyanate isomer I (2.72 g, 7 mmol) is heated in DMF at 120° C. for 5 hours.

The mixture is cooled, DMF evaporated off and acetic acid (25 ml) is added. The mixture is refluxed for 10 hours. The acetic acid is evaporated and the resulting mixture is purified on silica using chloroform/methanol as eluent.

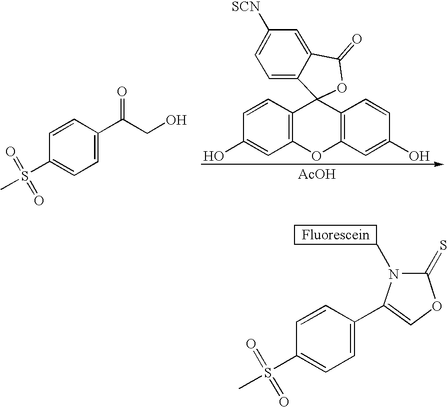

### Example 2

### Contrast Agent for Mapping of Steroid 5 Alpha-Reductase Activity

Pentasilylated epigallocatechin is prepared according to J. J. Plattner et al in JACS (1972) 94 8613-5.

Step 1.

5(6)-Carboxyfluorescein (1 mmol) is dissolved in DMF (30 ml). Dicyclohexylcarbodiimide (1.1 mmol) is added. The mixture is stirred at room temperature for 12 hours. A solution of pentasilylated epigallocatechin (1 mmol) and dimethylaminopyridine (50 mg) in DMF (5 ml) is added and the mixture is stirred for 48 hours at room temperature. The mixture is evaporated. The ester product is isolated by flash chromatography (silica, hexane/chloroform).

Step 2.

The pentasilylated epigallocatechin-fluorescein conjugate (0.5 mmol) is dissolved in tetrahydrofuran (30 ml). Triethylamine trihydrofluoride (1 mmol) is added and the mixture is stirred at ambient temperature for 12 hours. The mixture is evaporated and the carboxyfluorescein epigallocatechin conjugate is isolated by flash chromatography (silica, hexane, chloroform, methanol).

### Example 3

### Contrast Agent for Mapping of EGFR/erB2 Tyrosine Kinase

Step 1. N-[4-((3-bromophenyl)amino)quinazolin-7-y-]acrylamide is prepared according to J. B. Smaill et al J. Med. Chem. (1999) 42 1803-1815.

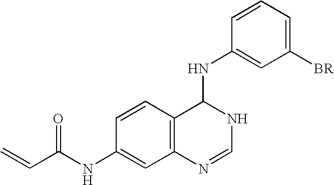

Step 2. N-[4-((3-bromophenyl)amino)quinazolin-7-y-]acrylamide from step 1 (1 mmol) and ethylenediamine (10 mmol) are dissolved in DMF (25 ml). The mixture is stirred at 50° C. for 12 hours. The solvent is evaporated off and the conjugate compound is isolated by flash chromatography (silica, hexane, chloroform, methanol).

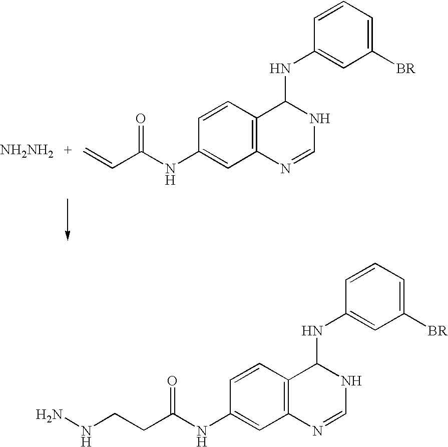

Step 3. Cy7-NHS ester (0.5 mmol), the conjugate compound from step 2 (0.5 mmol) and N-methylmorpholine (70 mg) are dissolved in DMF (30 ml). The mixture is stirred at 40° C. for 3 days. The Cy7 amide conjugate is isolated by flash chromatography (silica, hexane, ethyl acetate, methanol).

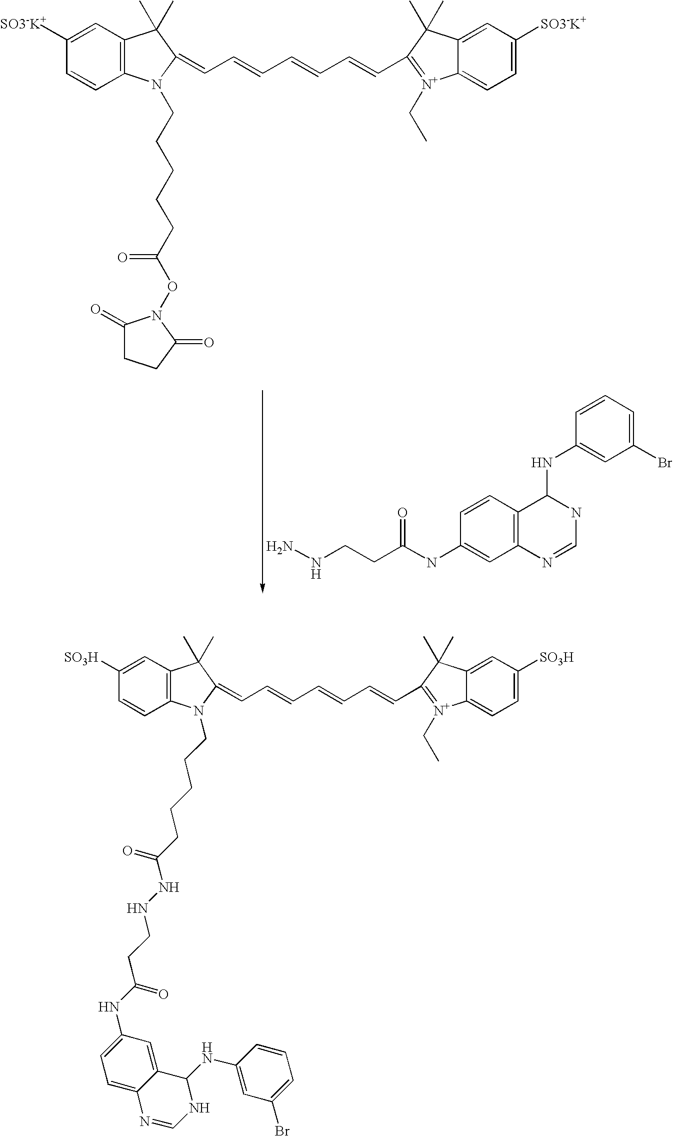

### Example 4

### Contrast Agent with Affinity for Androgen Receptor

Step 1.

Methyltrienolone (1 mmol) and 6-amino-1-hexanol (1 mmol) are dissolved in DMF (50 ml). The mixture is stirred at 100° C. for 24 hours. The solvent is removed and the Michael condensation product is isolated by chromatography (silica, hexan/ethyl acetate).

Step 2.

Cy 5.5 NHS ester (0.5 mmol), condensation product from step 1 above (0.5 mmol) and N-methylmorpholine (100 mg) are dissolved in DMF (30 ml). The mixture is stirred at 30° C. for 5 days. The Cy 5.5 ester with methyltrienolone (amino-hexanol spacer) is isolated by chromatography, silica, hexane/ethyl acetate).

### Example 5

### Contrast Agent with Affinity for Integrins: RGD Peptide Linked to Cy5.5

Step 1. Assembly of amino acids

The peptide sequence Asp-D-Phe-Lys-Arg-Gly was assembled on an Applied Biosystems 433A peptide synthesizer starting with 0.25 mmol Fmoc-Gly-SASRIN resin. An excess of 1 mmol pre-activated amino acids (using HBTU; O-Benzotriazol-1-yl-N,N,N′,N′-tetramethyluronium hexafluorophosohate) was applied in the coupling steps. The cleavage of the fully protected peptide from the resins was carried out by treatment of the resin with three portions of 35 mL of 1% trifluoroacetic acid (TFA) in dichloromethane (DCM) for 5 minutes each. The filtrates containing the peptide was immediately neutralised with 2% piperidine in DCM. The organics were extracted with water (3×100 mL), dried with MgSO₄ and evaporated in vacuo. Diethyl ether was added to the residue and the precipitate washed with ether and air-dried affording 30 mg of crude protected peptide. The product was analysed by analytical HPLC (conditions: Gradient, 20-70% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 7.58 min). Further product characterisation was carried out using electrospray mass spectrometry (MH⁺ calculated, 1044.5; MH⁺ found, 1044.4).

Step 2. N—C Cyclisation

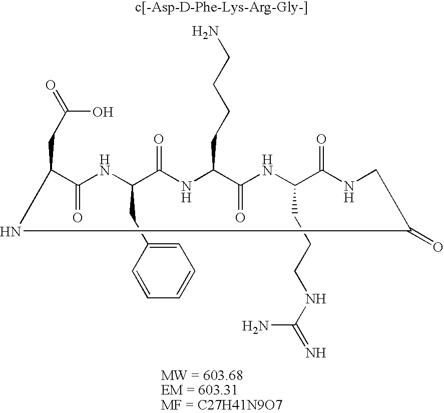

30 mg of the fully protected peptide, 16 mg of PyAOP, 4 mg of HOAt and 6 μL of N-methylmorpholine (NMM) were dissolved in dimethylformamide/DCM (1:1) and stirred over night. The mixture was evaporated in vacuo and diethyl ether added to the residue. The precipitate was washed with ether and air-dried. The crude cyclic fully protected peptide was treated with a solution of 25 mL TFA containing 5% water, 5% triisopropylsilane and 2.5% phenol for two hours. TFA was evaporated in vacuo and diethyl ether added to the residue. The precipitate was washed with ether and air-dried. Purification by preparative RP-HPLC (0-30% B over 40 min, where A=H₂O/0.1% TFA and B=CH₃CN/0.1% TFA, at a flow rate of 10 mL/min on a Phenomenex Luna 5μ, C18 250×21.20 mm column) of the crude material afforded 2.3 mg pure product peptide. The pure product was analysed by analytical HPLC (conditions: Gradient, 0-15% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 6.97 min). Further product characterisation was carried out using electrospray mass spectrometry (MH⁺ calculated, 604.3; MH⁺ found, 604.4).

Step 3. Conjugation of Cy5.5 to RGD Peptide

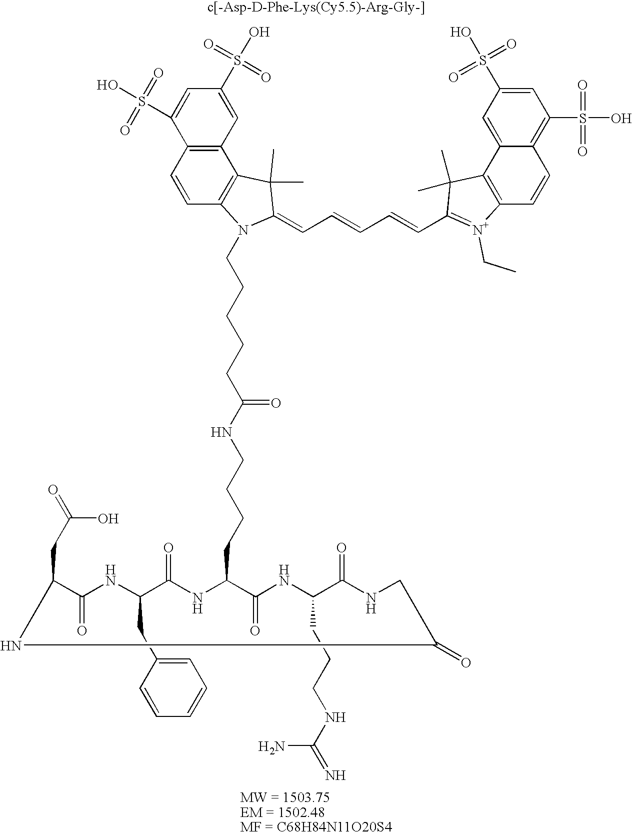

0.6 mg of the RGD peptide, 1.7 mg of Cy5.5 mono NHS ester and 5 μL of NMM were dissolved in 1 mL of dimethylformamide (DMF) and the reaction mixture stirred for 2 hrs. Diethyl ether was added to the DMF solution and the blue precipitate washed with diethyl ether and air-dried affording 0.7 mg of crude RGD peptide conjugated to Cy5.5.The pure product was analysed by analytical HPLC (conditions: Gradient, 5-50% B over 10 min where A=H₂O/0.1% TFA and B=CH₃CN/0.1% TFA; flow, 0.3 mL/min; column, Phenomenex Luna 3μ 5×2 mm; detection, UV 214 nm; product retention time 8.32 min). Further product characterisation was carried out using electrospray mass spectrometry (MH⁺ calculated, 1502.5; MH⁺ found, 1502.6).

### Example 6

### Synthesis of 3-[(4′-Fluorobiphenyl-4-sulfonyl)-(1-hydroxycarbamoylcyclopentyl)amino]propionic acid (Compound A) Derivatised with Cy5.5—Contrast Agent for Binding to MMP

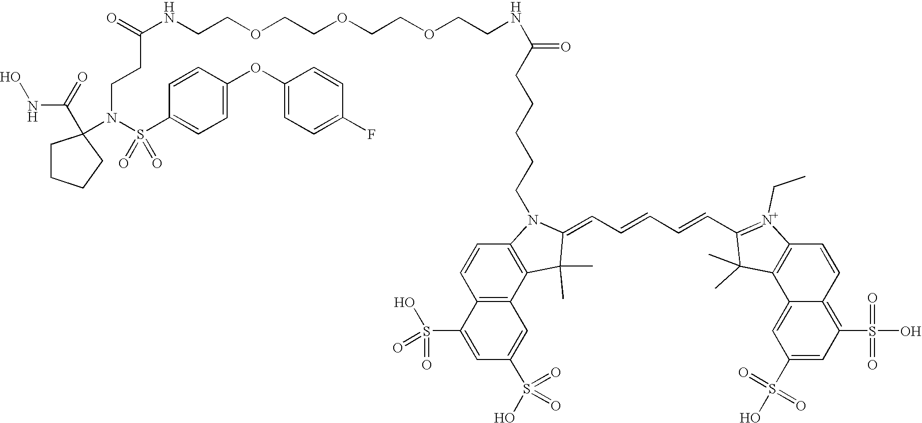

a) 1,11-Diazido-3,6,9-trioxaundecane

A solution of dry tetraethylene glycol (19.4 g, 0.100 mol) and methanesulphonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stirring was continued for 4 hrs. Water (60 ml) was added. To the mixture was added sodium hydrogencarbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

b) 11-Azido-3,6,9-trioxaundecanamine

To a mechanically, vigorously stirred suspension of 1,11-diazido-3,6,9-trioxaundecane (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0 g (88%) of yellow oil. Analysis by MALDI-TOF mass spectroscopy (matrix: □-cyano-4-hydroxycinnamic acid) gave a M+H peak at 219 as expected. Further characterisation using ¹H (500 MHz) and ¹³C (125 MHz) NMR spectroscopy verified the structure.

c) Linking Compound A to PEG(4)-N₃

To a solution of compound A (CP471358, Pfizer, 41 mg, 87μmol) in DMF (5 ml) were added 11-azido-3,6,9-trioxaundecanamine (19 mg, 87 μmol), HATU (Applied Biosystems, 33 mg, 87 μmol) and DIEA (Fluka, 30 μl, 174 μmol). After one hour reaction time the mixture was concentrated and the residue was purified by preparative HPLC (column Phenomenex Luna C18(2) 5 μm 21.2×250 mm, solvents: A=water/0.1% TFA and B=acetonitrile/0.1% TFA; gradient 30-60% B over 60 min; flow 10.0 ml/min, UV detection at 214 nm), giving 33.9 mg (59%) of product after lyophilisation. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.88 min with m/z 667.4 (MH⁺) as expected.

d) Synthesis of Compound A-PEG(4)-NH₂

To a solution of the PEG(4)-N₃ compound from c) (4.7 mg, 7.0 μmol) in methanol (4 ml) was added Pd/C (Koch-Light, ca 10 mg) added. The mixture was stirred at room temperature under hydrogen atmosphere (1 atm) for 10 min. The mixture was filtered and concentrated. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.17 min with m/z 641.4 (MH⁺) as expected. The product was used directly in the next step without further purification.

e) Conjugation of Cy 5.5

To a solution of the amine from d) (1.0 mg, 1.5 μmol) in DMF (0.2 ml) was added Cy 5.5-NHS (Amersham Biosciences, 1.0 mg, 1.0 μmol) and N-methylmorpholine (1 μl, 9 μmol). The reaction mixture was stirred for 48 h. MS analysis of the solution gave a spectrum showing starting material and the conjugated product at m/z 1539.7 (M⁺), expected 1539.4.

### Example 7

### Cy5-VEGF

Five micrograms of vascular endothelial growth factor (VEGF-121 cat. no. 298-VS/CF) (carrier-free, from R&D Systems) were dissolved in 19 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 646 nm, the emission being measured at 678 nm. The product was a fluorescent targeting molecule for the VEGF receptor.

### Example 8

### Cy5-TIMP-1

Five micrograms of tissue inhibitor of metalloproteinases-1 (TIMP-1, cat. no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 646 nm, the emission being measured at 678 nm. The product was a fluorescent targeting molecule for matrix metalloproteinases.

### Example 9

### Fluorescein-TIMP-1

Five micrograms of tissue inhibitor of metalloproteinases-1 (TIMP-1, cat. no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of fluorescein (Fluka), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 485 nm, the emission being measured at 538 nm. The product was a fluorescent targeting molecule for matrix metalloproteinases.

### Example 10

### Cy5-EGF

Sixty micrograms of epidermal growth factor (EGF, cat. no. 236-EG, 10 nmol) (from R&D Systems) were dissolved in 10 μl of 0.02 M borate buffer, pH 8.5. To this solution was added 10 μl buffer and 50 nmol of the N-hydroxysuccinimide ester of a carboxylic acid derivative of Cy5 (Amersham Biosciences). The reactive dye was dissolved in 5 μl of the same buffer, mixed 1:1 with dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was bright blue, fluoresced with excitation light at 646 nm, the emission being measured at 678 nm. The product was a fluorescent targeting molecule for the epidermal growth factor receptor.

### Example 11

### Cy7.5-EGF

Sixty micrograms of epidermal growth factor (EGF, cat. no. 236-EG, 10 nmol) (from R&D Systems) were dissolved in 10 μl of 0.02 M borate buffer, pH 8.5. To this solution was added 10 μl buffer and 50 nmol of the N-hydroxysuccinimide ester of a carboxylic acid derivative of Cy7.5 (Amersham Biosciences). The reactive dye was dissolved in 5 μl of the same buffer, mixed 1:1 with dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was dark green, fluoresced with excitation light at 700 nm, the emission being measured at 790 nm. The product was a fluorescent targeting molecule for the epidermal growth factor receptor.

### Example 12

### Fluorescein-EGF

Sixty micrograms of epidermal growth factor (EGF, cat. no. 236-EG, 10 nmol) (from R&D Systems) were dissolved in 10 μl of 0.02 M borate buffer, pH 8.5. To this solution was added 10 μl buffer and 50 nmol of the N-hydroxysuccinimide ester of a carboxylic acid derivative of fluorescein (Fluka), dissolved in 5 μl of dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was yellow, fluoresced with excitation light at 485 nm, the emission being measured at 538 nm. The product was a fluorescent targeting molecule for the epidermal growth factor receptor.

What is claimed is:

1. An optical imaging contrast agent with affinity for an abnormally expressed biological target associated with prostate cancer.

2. A contrast agent as claimed in claim 1 wherein the molecular weight is below 14 000 Daltons.

3. A contrast agent as claimed in claim 1 of formula I
V-L-R, (I)
wherein V is one or more vector moieties having affinity for an abnormally expressed target in prostate cancer, L is a linker moiety or a bond and R is one ore more reporter moieties detectable in optical imaging.

4. A contrast agent as claimed in claim 1 comprising a contrast agent substrate, wherein the target is an abnormally expressed enzyme, such that the contrast agent changes pharmacodynamic properties and/or pharmacokinetic properties upon a chemical modification from a contrast agent substrate to a contrast agent product upon a specific enzymatic transformation.

5. A contrast agent as claimed in claim 1 having affinity for any of the targets selected from steroid 5 alpha-reductase, fatty acid synthase, COX-2, prostate specific antigen, androgen receptor, lipoxygenase-5, VEGFR, Cyclin D1, CD44, Syndecan-1, prostate stem cell antigen (PSCA), Ki-67, alpha-methylacyl-CoA racemase (AMACR), cathepsin D, hepsin, MT1-MMP, urokinase receptor (uPAR), epidermal growth factor receptor (EGFR/her-2/neu), Fas (Apo-1/CD95), Fas ligand (FasL) and Macrophage Scavenger Receptor 1 (MSR1).

6. A contrast agent as claimed in claim 3 wherein V is selected from peptides, peptoid moieties, oligonucleotides, oligosaccharides, fat-related compounds and traditional organic drug-like small molecules.

7. A contrast agent as claimed in claim 3 wherein R is a dye that interacts with light in the wavelength region from the ultraviolet to the near-infrared part of the electromagnetic spectrum.

8. A pharmaceutical composition for optical imaging of prostate cancer, comprising a contrast agent as defined in claim 1 together with at least one pharmaceutically acceptable carrier or excipient.

9. The contrast agent as claimed in claim 1 wherein the contrast agent is used for the manufacture of a diagnostic agent.

10. A method of optical imaging of prostate cancer of an animate subject involving administering a contrast agent as defined in claim 1 to the subject and generating an optical image of at least a part of the subject to which said contrast agent has distributed.

11. The method as claimed in claim 10 for diagnosis of prostate cancer, for follow up of the progress of prostate cancer development, for follow up of treatment of prostate cancer or for surgical guidance.

12. The optical imaging contrast agent as defined in claim 1 wherein the contrast agent is used for optical imaging of prostate cancer.

13. (canceled)


SEQUENCE LISTING (1 sequence)

SEQ ID NO: 1 - protein, 5 aa, Artificial Sequence
  Synthetic peptide
DFKRG